(19) United States
(12) Patent Application Publication
(10) Pub. No.: US 2009/0143631 A1
(43) Pub. Date: Jun. 4, 2009

(54) Process For the Production of Olefins By Autothermal Cracking

(76) Inventors: Benjamin Patrick Gracey, Hull (GB); Ian Allan Reid, London (GB)

Correspondence Address: NIXON & VANDERHYE, PC, 901 NORTH GLEBE ROAD, 11TH FLOOR, ARLINGTON, VA, 22203, US

(21) Appl. No.: 11/795,837

(22) PCT Filed: Dec. 22, 2005
(86) PCT No.: PCT/GB2005/005005

§ 371 Date: Oct. 5, 2007

(30) Foreign Application Priority Data
Jan. 21, 2005 (GB) 0501253.9

(51) Int. Cl. C07C 4/02

(52) U.S. Cl. 585/648; 585/650

(57) ABSTRACT

The present invention provides a process for the production of olefins by autothermal cracking of a paraffinic hydrocarbon-containing feedstock in the presence of a molecular oxygen-containing gas, wherein said process comprises (a) providing a pre-heated, mixed feedstream comprising said paraffinic hydrocarbon containing feedstock and said molecular oxygen-containing gas, (b) subsequently mixing said pre-heated, mixed feedstream with a diluent, said diluent being pre-heated to a temperature of at least 400° C., to produce a diluted mixed feedstream comprising at least 10% by volume of diluent, and (c) contacting said diluted mixed feedstream with a catalyst capable of supporting combustion beyond the normal fuel rich limit of flammability, to provide a hydrocarbon product stream comprising olefins.

The present invention relates to a process for the production of olefins. In particular, the present invention relates to a process for the production of olefins by autothermal cracking.

Autothermal cracking is a route to olefins in which the hydrocarbon feed is mixed with oxygen and passed over an autothermal cracking catalyst. The autothermal cracking catalyst is capable of supporting combustion beyond the fuel rich limit of flammability. Combustion is initiated on the catalyst surface and the heat required to raise the reactants to the process temperature and to carry out the endothermic cracking process is generated in situ. Generally the hydrocarbon feed and molecular oxygen are passed over a supported catalyst to produce the olefin product. Typically, the catalyst comprises at least one platinum group metal, for example, platinum. The autothermal cracking process is described in EP 332289B; EP-529793B; EP-A-0709446 and WO 00/14035.

It is known that additional feed components may also be passed to the autothermal cracker. Suitable additional feed components include, for example, hydrogen and steam. Hydrogen, for example, is typically fed because it reacts preferentially with oxygen to generate the heat required for autothermal cracking of the hydrocarbon feed, reducing the requirement to burn the more valuable hydrocarbon feed to generate said heat.

We have now found that the autothermal cracking of hydrocarbons may be advantageously operated by diluting a pre-mixed and pre-heated hydrocarbon and molecular oxygen-containing feed with a suitable pre-heated diluent prior to contact with the catalyst.

Hence, in a first aspect, the present invention provides a process for the production of olefins by autothermal cracking of a paraffinic hydrocarbon-containing feedstock in the presence of a molecular oxygen-containing gas, wherein said process comprises
(a) providing a pre-heated, mixed feedstream comprising said paraffinic hydrocarbon-containing feedstock and said molecular oxygen-containing gas,
(b) subsequently mixing said pre-heated, mixed feedstream with a diluent, said diluent being pre-heated to a temperature of at least 400° C., to produce a diluted mixed feedstream comprising at least 10% by volume of diluent, and
(c) contacting said diluted mixed feedstream with a catalyst capable of supporting combustion beyond the normal fuel rich limit of flammability, to provide a hydrocarbon product stream comprising olefins.

Step (a) of the process of the present invention comprises providing a pre-heated, mixed feedstream comprising paraffinic hydrocarbon-containing feedstock and molecular oxygen-containing gas. The pre-heated, mixed feedstream may be produced by any suitable method, but, especially at pressures where flammability constraints may be more significant, is preferably produced by:
(i) separately pre-heating said paraffinic hydrocarbon-containing feedstock and said molecular oxygen-containing gas, and
(ii) mixing the pre-heated paraffinic hydrocarbon-containing feedstock and pre-heated molecular oxygen-containing gas to produce said pre-heated, mixed feedstream.

The paraffinic hydrocarbon-containing feedstock and the molecular oxygen-containing gas may be pre-heated to any suitable temperatures before mixing with each other. Advantageously, one or more heat exchangers may be employed to pre-heat the paraffinic hydrocarbon-containing feedstock and molecular oxygen-containing gas prior to mixing. Generally, the amount of pre-heating that can be performed is limited to temperatures wherein the pre-heated, mixed feedstream will be below the autoignition temperature of the mixture. This is usually significantly below the reaction temperature obtained when the mixed feedstream contacts the catalyst.

Typically, the paraffinic hydrocarbon feedstock is pre-heated to less than 300° C.

Typically, the molecular oxygen-containing is pre-heated to less than 150° C., preferably less than 100° C.

Typically, the pre-heated, mixed feedstream will be at a temperature of less than 300° C.

Preferably the pre-heated mixed feedstream comprises paraffinic hydrocarbon-containing feedstock and molecular oxygen-containing gas at a ratio of paraffinic hydrocarbon to molecular oxygen-containing gas of 5 to 16 times, preferably 5 to 13.5 times, more preferably 6 to 10 times, the stoichiometric ratio of paraffinic hydrocarbon to molecular oxygen-containing gas required for complete combustion of the hydrocarbon to carbon dioxide and water.

Hydrogen (molecular hydrogen) may be co-fed to the process of the present invention as a component of the pre-heated mixed feedstream also comprising paraffinic hydrocarbon-containing feedstock and molecular oxygen-containing gas. Suitably, the molar ratio of hydrogen to molecular oxygen-containing gas is in the range 0.2 to 4, preferably, in the range 1 to 3. Preferably, hydrogen is pre-mixed with the paraffinic hydrocarbon-containing feedstock before mixing with the molecular oxygen-containing gas to produce a pre-heated, mixed feedstream.

In step (b) of the process of the present invention, the mixed feedstream is mixed with a diluent, said diluent being pre-heated to a temperature of at least 400° C., to produce a diluted mixed feedstream comprising at least 10% by volume of diluent.

A heat exchanger may be employed to pre-heat the diluent prior to mixing.

Typically, the diluted mixed feedstream comprises 20 to 70% by volume of diluent, such as 40 to 50% by volume.

The diluent may be pre-heated to at least 600° C., such as at least 700° C.

Preferably, the diluted mixed feedstream produced will be at a temperature of at least 400° C., such as at least 500° C.

The diluent may be a single material or may comprise a mixture of materials.

The diluent comprises at least 80% by volume, such as at least 90% by volume, of materials (hereinafter diluent materials) other than hydrogen, molecular oxygen and paraffinic hydrocarbon, with the exception that where the paraffinic hydrocarbon-containing feedstock comprises paraffinic hydrocarbons having at least two carbon atoms then the diluent materials may also comprise methane.

Preferred diluent materials are materials which are inert in the process of the present invention. Because methane is significantly less reactive than paraffinic hydrocarbons having at least two carbon atoms, such as ethane and propane, when the process of the present invention is conducted with a paraffinic hydrocarbon-containing feedstock comprising paraffinic hydrocarbons having at least two carbon atoms the conversion of methane, if any, is relatively low. Thus, even if methane is not completely inert under the conditions used, the conversion of methane is typically less than 10% of any methane contacted with the catalyst in step (c).

Alternatively, or in addition to any inert materials and/or methane, the diluent materials may be materials (other than hydrogen and molecular oxygen) which are unreactive to produce olefins. By “materials unreactive to produce olefins” as used herein is meant diluent materials which may undergo chemical changes in the autothermal cracking process, and hence are not inert, but the (direct) products from such changes are not olefins. An example is carbon monoxide.

Most preferably, the diluent comprises at least 80% by volume, preferably at least 90% by volume, of steam, carbon monoxide, carbon dioxide, an inert gas, such as helium, neon, argon or nitrogen, methane (where the paraffinic hydrocarbon-containing feedstock comprises paraffinic hydrocarbons having at least two carbon atoms), or a mixture thereof.

Methane and carbon monoxide, for example, may be obtained as by-products from the autothermal cracking process of step (c).

The diluent is mixed with the mixed feedstream immediately before the diluted mixed feedstream contacts the catalyst, in particular within 100 ms. Preferably, the diluted mixed feedstream is contacted with the catalyst within 50 milliseconds of the diluent being mixed with the pre-heated mixed feedstream, and more preferably within 10 ms. For avoidance of doubt this time is measured from the time of first contact of diluent with the pre-heated mixed feedstream.

The mixing and rapid contact with the catalyst is achieved by providing a suitable source for the diluent located relatively close to the surface of the catalyst bed and/or to any catalyst holder.

The diluent may be mixed with the mixed feedstream using any suitable mixing device. One such device that may be used is a diffusion-bonded block formed by diffusion bonding of layers of etched metal structures. Such structures are known for heat exchange uses, and are described generally, for example, in “Industrial Microchannel Devices—Where are we Today ?”; Pua, L. M. and Rumbold, S. O.; First International Conferences on Microchannels and Minichannels, Rochester, N.Y., April 2003.

A preferred method of introducing the diluent is by use of a sparger having at least 4 outlets distributed close to the top face of the catalyst (or catalyst holder).

Because of its high temperature and because of the position of introduction of the diluent (immediately before the catalyst and after mixing of the paraffinic hydrocarbon-containing feedstock and molecular oxygen-containing gas), the diluent may advantageously be used to introduce quantities of other hydrocarbons (being hydrocarbons other than methane or the paraffinic hydrocarbons which are the principle components of the paraffinic hydrocarbon-containing feedstock) to the process of the present invention. Hence, the diluent may also comprise up to 20% by volume of hydrocarbons other than methane or the paraffinic hydrocarbons which are the principle components of the paraffinic hydrocarbon-containing feedstock, for example of dienes, such as butadiene and/or of “heavier” hydrocarbons, which are generally hydrocarbons which are liquids at room temperature and pressure.

The process of the present invention allows such hydrocarbons to be delivered to the reaction at high temperature, for example reducing difficulties with feeding heavy hydrocarbons as liquids.

The diluent may also be used to deliver quantities of hydrogen at high temperature to the reaction, and hence the diluent may comprise up to 20% by volume of hydrogen.

Alternatively, in the absence of hydrocarbons or hydrogen in the diluent, the diluent may comprise up to 20% by volume of molecular oxygen.

A most preferred diluent comprises steam, such as 20 to 100% by volume, preferably 50 to 100% by volume of steam.

Steam has the added advantage that it will inhibit formation of pyrolytic carbon on the catalyst and the formation of acetylenes in the cracking reaction.

In one embodiment, the pre-heated diluent comprising steam may be produced by providing a stream comprising hydrogen and molecular oxygen, which react to produce steam (water) and generate the heat required to heat the stream to the required pre-heat temperature.

In an alternative embodiment, the pre-heated diluent comprising steam may be produced by providing a stream comprising methane (and optionally hydrogen) and reacting this with molecular oxygen, to produce a hot stream comprising steam (water), carbon dioxide and, optionally, any unreacted methane, at least some of which is used as the pre-heated diluent.

The hot stream comprising steam produced from hydrogen and molecular oxygen or steam, carbon dioxide and any unreacted methane produced from methane and molecular oxygen is typically initially at a temperature of much higher than 400° C. and, hence, much higher than that required for the diluent stream. The stream may be cooled by heat exchange and/or diluted to produce the diluent stream of the desired temperature. Where the stream is cooled by heat exchange the heat removed may be used as pre-heat for other feeds to the process, such as the paraffinic hydrocarbon-containing feedstock and/or the molecular oxygen-containing gas.

Preferably, where steam is used as the diluent at least some of the steam may be obtained from downstream processing steps, such as from the quench used to cool the reaction products from the autothermal cracking process.

In general, the dilution of the mixed feedstream by the diluent allows the reaction to be operated at relatively low partial pressures of the paraffinic hydrocarbon-containing feedstock (compared to the total pressure), which can lead to improved selectivity. A lower partial pressure of paraffinic hydrocarbon-containing feedstock will also lead to a reduced partial pressure of products in the product stream, which will reduce further reactions taking place in the product stream, and hence reduce the quench requirements for the product stream. The dilution of the mixed feedstream by the diluent also allows higher flow rates to be used which can make feeding of liquid paraffinic hydrocarbon-containing streams to the catalyst easier.

The use of a hot diluent reduces the heating requirements of the mixed feedstream compared to addition of a cold diluent. The use of a hot diluent which is mixed with a mixed (hydrocarbon and molecular oxygen-containing) feedstream to produce a diluted mixed feedstream immediately before the diluted mixed feedstream contacts the catalyst allows a significant amount of heat to be introduced to the reaction mixture with significantly reduced flammability issues compared to if the hot diluent were introduced earlier in the mixing process (when the residence time of the diluted mixed feedstream may exceed the ignition delay time for a particular feedstream), allowing a higher temperature diluted mixed feed to be obtained. Mixing the hot diluent immediately before the diluted mixed feedstream contacts the catalyst also reduces opportunities for heat loss from the mixed stream, improving the efficiency of the heat introduction. Where the diluent is fed at above the reaction temperature the feeding of the hot diluent leads to a reduction in the amount of feed that has to be combusted to generate heat for cracking (compared to the absence of a diluent), and can lead to significant increases in the yield of olefins obtainable. In one embodiment, the process may also be operated without hydrogen co-feed to the process or at least with reduced hydrogen than normally required (hydrogen being fed via the pre-heated mixed feedstream and/or as part of the diluent).

The use of a hot diluent also has advantages in the start-up and shut-down of the autothermal cracking reaction. During start-up, the hot diluent can be introduced to the catalyst before the reactants, causing the catalyst to be pre-heated to the temperature of the diluent. When the reactants are introduced the catalyst rapidly heats to reaction temperature, which is typically in the range 600° C. to 1200° C. at the exit of the catalyst. Because the catalyst is already at a higher temperature from use of hot diluent prior to introduction of the reactants, the thermal stresses across the catalyst on initiation of reaction are reduced.

Similarly, on shut-down, the thermal stresses across the catalyst can be reduced by using the hot diluent, optionally with a purge gas such as nitrogen, rather than the purge gas alone.

In step (c) of the present invention the diluted mixed feedstream is contacted with a catalyst capable of supporting combustion beyond the normal fuel rich limit of flammability, to provide a hydrocarbon product stream comprising olefins.

The catalyst capable of supporting combustion beyond the fuel rich limit of flammability usually comprises a Group VIII metal as its catalytic component. Suitable Group VIII metals include platinum, palladium, ruthenium, rhodium, osmium and iridium. Rhodium, and more particularly, platinum and palladium are preferred. Typical Group VIII metal loadings range from 0.01 to 100 wt %, preferably, between 0.01 to 20 wt %, and more preferably, from 0.01 to 10 wt % based on the total dry weight of the catalyst.

The reaction may suitably be carried out at a catalyst exit temperature in the range 600° C. to 1200° C., preferably, in the range 850° C. to 1050° C. and, most preferably, in the range 900° C. to 1000° C.

The process of the present invention may be used to convert both liquid and gaseous paraffinic hydrocarbons into olefins. Suitable liquid hydrocarbons include naphtha, gas oils, vacuum gas oils and mixtures thereof. Suitable gaseous hydrocarbons include ethane, propane, butane and mixtures thereof.

When used to convert gaseous hydrocarbons the process of the present invention is preferably operated at an elevated pressure of at least 5 barg (total pressure of diluted mixed feedstream), most preferably in the range 10 to 40 barg, for example, in the range 10 to 30 barg. When used to convert gaseous hydrocarbons, the process of the present invention is preferably operated at a partial pressure of paraffinic hydrocarbon-containing feedstock and molecular oxygen containing gas in the diluted mixed feedstream of greater than 2 barg, such as in the range 5 to 25 barg and advantageously in the range 10 to 18 barg.

When used to convert liquid hydrocarbons, the process of the present invention is preferably operated at an elevated pressure of at least 1 barg (total pressure of diluted mixed feedstream), most preferably in the range 1 to 5 barg. When used to convert liquid hydrocarbons, the process of the present invention is preferably operated at a partial pressure of paraffinic hydrocarbon-containing feedstock and molecular oxygen containing gas in the diluted mixed feedstream of greater than 0.5 barg, such as in the range 0.5 to 4 barg.

Any suitable molecular oxygen-containing gas may be used. Suitably, the molecular oxygen-containing gas is molecular oxygen, air and/or mixtures thereof. The molecular oxygen-containing gas may be mixed with an inert gas such as nitrogen or argon.

The diluted mixed feedstream is passed over the catalyst at a gas hourly space velocity which is pressure dependent and typically greater than 10,000 h⁻¹ barge⁻¹, preferably greater than 20,000 h⁻¹ barge⁻¹ and, most preferably, greater than 100,000 h⁻¹ barge⁻¹. For example, at 20 barg pressure, the gas hourly space velocity is most preferably, greater than 2,000,000 h⁻¹. It will be understood, however, that the optimum gas hourly space velocity will depend upon the nature of the feed composition.

The reaction products are preferably quenched with water as they emerge from the autothermal cracker, typically in a suitable quench tower.

To avoid further reactions taking place, usually the product stream is cooled to between 750-600° C. within 100 milliseconds of formation, preferably within 50 milliseconds of formation and most preferably within 20 milliseconds of formation. As noted previously, the use of a diluent according to the process of the present invention reduces the rate of further reactions taking place in the product stream compared to reactions in the absence of diluent. The present invention therefore provides the potential to eliminate the direct quench and replace it with more “conventional” heat recovery systems, such as a waste heat boiler.

Where a quench is present, and wherein the autothermal cracking process is operated at a partial pressure of 5-20 barg usually the products are quenched and the temperature cooled to between 750-600° C. within 20 milliseconds of formation.

Where a quench is present, and wherein the autothermal cracking process is operated at a partial pressure of greater than 20 barg the products are quenched and the temperature cooled to between 750-600° C. within 10 milliseconds of formation.

The hydrocarbon product stream, in addition to olefins, may comprise unreacted paraffinic hydrocarbons, hydrogen, carbon monoxide, methane, and small amounts of acetylenes, aromatics and carbon dioxide, which need to be separated from the desired olefins.

Where a Group VIII catalyst is employed, it is preferably employed in combination with a catalyst promoter. The promoter may be a Group IIIA, IVA, and/or VA metal. Alternatively, the promoter may be a transition metal; the transition metal promoter being a different metal to that which may be employed as the Group VIII transition metal catalytic component.

Preferred Group IIIA metals include Al, Ga, In and Tl. Of these, Ga and In are preferred. Preferred Group IVA metals include Ge, Sn and Pb. Of these, Ge and Sn are preferred. The preferred Group VA metal is Sb. The atomic ratio of Group VIII B metal to the Group IIIA, IVA or VA metal may be 1:0.1-50.0, preferably, 1:0.1-12.0.

Suitable metals in the transition metal series include those metals in Group IB to VIII of the Periodic Table. In particular, transition metals selected from Groups IB, IIB, VIB, VIIB and VIII of the Periodic Table are preferred. Examples of such metals include Cr, Mo, W, Fe, Ru, Os, Co, Rh, Ir, Ni, Pt, Cu, Ag, Au, Zn, Cd and Hg. Preferred transition metal promoters are Mo, Rh, Ru, Ir, Pt, Cu and Zn. The atomic ratio of Group VIII metal to transition metal promoter may be 1:0.1-50.0, preferably, 1:0.1-12.0.

Preferably, the catalyst comprises only one promoter; the promoter being selected from Group IIIA, Group IVA, Group VB and the transition metal series. For example, the catalyst may comprise a metal selected from rhodium, platinum and palladium and a promoter selected from the group consisting of Ga, In, Sn, Ge, Ag, Au or Cu. Preferred examples of such catalysts include Pt/Ga, Pt/In, Pt/Sn, Pt/Ge, Pt/Cu, Pd/Sn, Pd/Ge, Pd/Cu and Rh/Sn. The Rh, Pt or Pd may comprise between 0.01 and 5.0 wt %, preferably, between 0.01 and 2.0 wt %, and more preferably, between 0.05 and 1.0 wt % of the total weight of the catalyst. The atomic ratio of Rh, Pt or Pd to the Group IIIA, IVA or transition metal promoter may be 1:0.1-50.0, preferably, 1:0.1-12.0. For example, atomic ratios of Rh, Pt or Pd to Sn may be 1:0.1 to 50, preferably, 1:0.1-12.0, more preferably, 1:0.2-3.0 and most preferably, 1:0.5-1.5. Atomic ratios of Pt or Pd to Ge, on the other hand, may be 1:0.1 to 50, preferably, 1:0.1-12.0, and more preferably, 1:0.5-8.0. Atomic ratios of Pt or Pd to Cu may be 1:0.1-3.0, preferably, 1:0.2-2.0, and more preferably, 1:0.5-1.5.

Alternatively, the promoter may comprise at least two metals selected from Group IIIA, Group IVA and the transition metal series. For example, where the catalyst comprises platinum, the platinum may be promoted with two metals from the transition metal series, for example, palladium and copper. Such Pt/Pd/Cu catalysts may comprise palladium in an amount of 0.01 to 5 wt %, preferably, 0.01 to 2 wt %, and more preferably, 0.01 to 1 wt % based on the total weight of the dry catalyst. The atomic ratio of Pt to Pd may be 1:0.1-10.0, preferably, 1:0.5-8.0, and more preferably, 1:1.0-5.0. The atomic ratio of platinum to copper is preferably 1:0.1-3.0, more preferably, 1:0.2-2.0, and most preferably, 1:0.5-1.5.

Where the catalyst comprises platinum, it may alternatively be promoted with one transition metal, and another metal selected from Group IIIA or Group IVA of the periodic table. In such catalysts, palladium may be present in an amount of 0.01 to 5 wt %, preferably, 0.01 to 2.0 wt %, and more preferably, 0.05-1.0 wt % based on the total weight of the catalyst. The atomic ratio of Pt to Pd may be 1:0.1-10.0, preferably, 1:0.5-8.0, and more preferably, 1:1.0-5.0. The atomic ratio of Pt to the Group IIIA or IVA metal may be 1:0.1-60, preferably, 1:0.1-50.0. Preferably, the Group IIIA or IVA metal is Sn or Ge, most preferably, Sn.

For the avoidance of doubt, the Group VIII metal and promoter in the catalyst may be present in any form, for example, as a metal, or in the form of a metal compound, such as an oxide.

The catalyst may be unsupported, such as in the form of a metal gauze, but is preferably supported. Any suitable support material may be used, such as ceramic or metal supports, but ceramic supports are generally preferred. Where ceramic supports are used, the composition of the ceramic support may be any oxide or combination of oxides that is stable at high temperatures of, for example, between 600° C. and 1200° C. The support material preferably has a low thermal expansion co-efficient, and is resistant to phase separation at high temperatures.

Suitable ceramic supports include corderite, lithium aluminium silicate (LAS), alumina (α-Al₂O₃), yttria stabilised zirconia, alumina titanate, niascon, and calcium zirconyl phosphate. The ceramic supports may be wash-coated, for example, with γ-Al₂O₃.

The support is preferably in the form of a foam or a honeycomb monolith.

The catalyst capable of supporting combustion beyond the fuel rich limit of flammability may be prepared by any method known in the art. For example, gel methods and wet-impregnation techniques may be employed. Typically, the support is impregnated with one or more solutions comprising the metals, dried and then calcined in air. The support may be impregnated in one or more steps. Preferably, multiple impregnation steps are employed. The support is preferably dried and calcined between each impregnation, and then subjected to a final calcination, preferably, in air. The calcined support may then be reduced, for example, by heat treatment in a hydrogen atmosphere.

1-10. (canceled)

11. A process for the production of olefins by autothermal cracking of a paraffinic hydrocarbon-containing feedstock in the presence of a molecular oxygen-containing gas, wherein said process comprises
(a) providing a pre-heated, mixed feedstream comprising said paraffinic hydrocarbon-containing feedstock and said molecular oxygen-containing gas,
(b) subsequently mixing said pre-heated, mixed feedstream with a diluent, said diluent being pre-heated to a temperature of at least 400° C., to produce a diluted mixed feedstream comprising at least 10% by volume of diluent, and
(c) contacting said diluted mixed feedstream with a catalyst capable of supporting combustion beyond the normal fuel rich limit of flammability, to provide a hydrocarbon product stream comprising olefins,
wherein the diluted mixed feedstream is contacted with the catalyst within 100 milliseconds of the diluent being mixed with the pre-heated mixed feedstream.

12. A process as claimed in claim 11, wherein the pre-heated mixed feedstream comprises paraffinic hydrocarbon-containing feedstock and molecular oxygen-containing gas at a ratio of paraffinic hydrocarbon to molecular oxygen-containing gas of 5 to 16 times the stoichiometric ratio of hydrocarbon to molecular oxygen-containing gas required for complete combustion of the hydrocarbon to carbon dioxide and water.

13. A process as claimed in claim 11, wherein the diluted mixed feedstream comprises 20 to 70% by volume of diluent, such as 40 to 50% by volume.

14. A process as claimed in claim 11, wherein the diluent comprises at least 80% by volume or inert materials, materials other than hydrogen and molecular oxygen which are unreactive to produce olefins and, where the paraffinic hydrocarbon-containing feedstock comprises paraffinic hydrocarbons having at least two carbon atoms, methane.

15. A process as claimed in claim 14, wherein the diluent comprises steam, carbon monoxide, carbon dioxide, an inert gas, such as helium, argon or nitrogen, methane (where the paraffinic hydrocarbon-containing feedstock comprises paraffinic hydrocarbons having at least two carbon atoms) or a mixture thereof.

16. A process as claimed in claim 15, wherein the diluent comprises steam.

17. A process as claimed in claim 11, wherein the diluted mixed feedstream is contacted with the catalyst within 10 milliseconds of the diluent being mixed with the pre-heated mixed feedstream.

18. A process as claimed in claim 14, wherein the diluent also comprises up to 20% by volume of hydrocarbons other than methane or the hydrocarbon which is the principle component of the paraffinic hydrocarbon-containing feedstock.

19. A process as claimed in claim 11, wherein the catalyst capable of supporting combustion beyond the fuel rich limit of flammability usually comprises a Group VIII metal as its catalytic component.

20. A process as claimed in claim 11, wherein the paraffinic hydrocarbon-containing feedstock comprises ethane, propane, butane or mixtures thereof.
